(19) United States
(12) Patent Application Publication
(10) Pub. No.: US 2020/0022682 A1
(43) Pub. Date: Jan. 23, 2020

(54) Ultrasonic Diagnosis Device and Electronic Circuit

(71) Applicant: Hitachi, Ltd., Tokyo (JP)

(72) Inventors: Takuya KANEKO, Tokyo (JP); Shinya KAJIYAMA, Tokyo (JP)

(21) Appl. No.: 16/361,887

(22) Filed: Mar. 22, 2019

(30) Foreign Application Priority Data
Jul. 19, 2018 (JP) 2018-135702

(51) Int. Cl. A61B 8/00; A61B 8/08; G01S 7/52

(52) U.S. Cl. CPC A61B 8/54; A61B 8/52; G01S 7/52077; A61B 8/4444; G01S 7/5208; A61B 8/565

(57) ABSTRACT

An electronic circuit in an ultrasonic probe includes a plurality of sub beamformers and a control unit. Each sub beamformer includes M delay circuits and an adding circuit. Each delay circuit includes a memory cell array which is formed of N memory cells. Conditions of cyclic operations of the M memory cell arrays (for example, timings of start triggers) are made irregular, such that use starting stage numbers in the M memory cell arrays are different.

### BACKGROUND OF THE INVENTION

### 1. Field of the Invention

The present invention relates to an ultrasonic diagnosis device and an electronic circuit, and more particularly, to delay processing in an ultrasonic probe.

### 2. Description of Related Art

An ultrasonic probe (3D probe) provided with a two dimensional vibration element array is used to obtain volume data by two-dimensionally scanning with an ultrasonic beam. In general, an electronic circuit is installed in the 3D probe, and the electronic circuit includes a plurality of sub beamformers to execute sub beamforming. Each sub beamformer includes a plurality of delay circuits and an adding circuit, and, after a plurality of reception signals are delay-processed in the plurality of delay circuits, the plurality of reception signals after being delay-processed are added at the adding circuit. The plurality of reception signals output from the plurality of sub beamformers are output to a device main body. In the above description, processing when signals are received has been described, but, when signals are transmitted, sub beamforming is executed if necessary.

Each of the delay circuits in each sub beamformer includes, for example, a memory cell array (see Japanese Patent No. 6205481 and Chao Chen, et al., A Front-End ASIC With Receive Sub-array Beamforming Integrated With a 32×32 PZT Matrix Transducer for 3-D Transesophageal Echocardiography, IEEE Journal of Solid-State Circuits, Vol. 52, No. 4, 2017 (Non-Patent Literature 1)). Each memory cell constituting the memory cell array acts as a sample and hold circuit. The memory cell array is cyclically used like a ring memory. In other words, the memory cell array performs a cyclic operation.

In the plurality of memory cell arrays in the sub beamformer, the same noise may be mixed with a plurality of memory cells belonging to a specific stage number, or the same noise may occur in such plurality of memory cells. Such a noise may periodically occur due to the cyclic operation of the memory cell array, and may increase at the step of adding the plurality of reception signals, thereby degrading an S/N ratio.

Non-Patent Literature 1 discloses a technique for dispersing noise generation timing by additionally arranging one extension memory cell in a memory cell array and selecting use and non-use of the extension memory cell. When such a configuration is employed, another problem that the number of memory cells increases arises.

### SUMMARY OF THE INVENTION

An object of the present invention is to eliminate or reduce a noise resulting from a plurality of memory cell arrays. Alternatively, an object of the present invention is to eliminate or reduce a noise resulting from a plurality of memory cell arrays, while avoiding an increase in the number of memory cells.

An ultrasonic diagnosis device according to the present invention includes: M delay circuits configured to delay M reception signals, each delay circuit including a memory cell array which is formed of N memory cells from a first stage to an N-th stage, the M delay circuits operating in parallel, an adding circuit configured to add the M reception signals output from the M delay circuits; and a control unit configured to control a cyclic operation of the memory cell array according to a set delay time in each delay circuit, and conditions of the cyclic operations of the M memory cell arrays are made irregular, such that use starting stage numbers in the M memory cell arrays included in the M delay circuits are different.

An electronic circuit according to the present invention includes: M delay circuits installed in an ultrasonic probe to delay M reception signals, each delay circuit including a memory cell array which is formed of N memory cells from a first stage to an N-th stage, the M delay circuits operating in parallel; an adding circuit installed in the ultrasonic probe to add the M reception signals output from the M delay circuits; and a control unit installed in the ultrasonic probe to control a cyclic operation of the memory cell array according to a set delay time in each of the delay circuits, and conditions of the cyclic operations of the M memory cell arrays are made irregular, such that use starting stage numbers in the M memory cell arrays included in the M delay circuits are different.

According to the present invention, a noise resulting from a plurality of memory cell arrays can be eliminated or reduced. Alternatively, according to the present invention, a noise resulting from a plurality of memory cell arrays can be eliminated or reduced, while avoiding an increase in the number of memory cells.

### BRIEF DESCRIPTION OF THE DRAWINGS

FIG. 1 is a block diagram illustrating an ultrasonic diagnosis device according to an embodiment;

FIG. 2 is a circuit diagram illustrating a delay circuit;

FIG. 3 is a view to explain generation of a periodic noise;

FIG. 4 is a view illustrating a result of suppressing a periodic noise;

FIG. 5 is a block diagram illustrating a first embodiment;

FIG. 6 is a timing chart illustrating the first embodiment;

FIG. 7 is a block diagram illustrating a second embodiment;

FIG. 8 is a timing chart illustrating the second embodiment;

FIG. 9 is a timing chart illustrating a phenomenon that can occur in the first embodiment;

FIG. 10 is a timing flowchart illustrating a variation of the first embodiment;

FIG. 11 is a concept view illustrating a third embodiment;

FIG. 12 is a block diagram illustrating the third embodiment;

FIG. 13 is a block diagram illustrating a first example of a shift circuit in the third embodiment; and

FIG. 14 is a block diagram illustrating a second example of the shift circuit in the third embodiment.

### DESCRIPTION OF EMBODIMENTS

Hereinafter, embodiments will be described based on the drawings.

### (1) Summary of Embodiments

An ultrasonic diagnosis device according to an embodiment includes M delay circuits, an adding circuit, and a control unit. Each delay circuit includes a memory cell array which is formed of N memory cells from a first stage to an N-th stage. The adding circuit adds M reception signals output from the M delay circuits. The control unit controls a cyclic operation of the memory cell array according to a set delay time for each delay circuit. Conditions of the cyclic operations of the M memory cell arrays are made irregular, such that use starting stage numbers in the M memory cell arrays included in the M delay circuits are different. Accordingly, since output timings of noises from the respective memory cell arrays become irregular, an increase of a noise in the adding circuit is avoided. According to the above-described configuration, there is an advantage that there is no need to increase the number of memory cells to suppress the noise.

M and N are integers greater than or equal to 2, respectively. In an embodiment, the M memory cell arrays included in the M delay circuits are synchronized with one another and operate in parallel. Each of the memory cell arrays cyclically operates like a ring memory. However, in an embodiment, each memory cell may be configured with an analogue memory element, and the memory cell array functions as a random access memory.

In an embodiment, in each delay circuit, the cyclic operation of the memory cell array starts according to a start trigger, and the control unit makes timings of M start triggers for regulating the operations of the M delay circuits irregular. By making the timings of the start triggers irregular, the use starting stage numbers between the plurality of memory cell arrays become irregular. Accordingly, an increase of a noise at the adding step is suppressed.

The control unit may correct the timings of the M start triggers according to a delay time set in each delay circuit. Even if the timing of the start trigger is controlled, the use starting stage numbers between the plurality of memory cell arrays may become regular according to the delay time. Such a situation can be avoided by correcting the timing of the start trigger.

In an embodiment, in each delay circuit, the cyclic operation of the memory cell array starts from a memory cell of a stage number corresponding to an offset, and the control unit makes M offsets given to the M delay circuits irregular. By making the offsets irregular, the use starting stage numbers between the plurality of memory cell arrays become irregular. Accordingly, an increase of a noise at the adding step is suppressed.

The control unit may correct the M offsets according to the delay time set in each delay circuit. Even if the offset is controlled, the use starting stage numbers may become regular in part according to the delay time. Such a situation can be avoided by correcting the timing of the start trigger.

In an embodiment, the control unit includes a generation circuit which is installed in each delay circuit to generate N control signals to be applied to the N memory cells, and a wiring change circuit which changes wirings of the N control signals in each delay circuit that is required to change wiring from among the M delay circuits, and outputs the N control signals after wiring change. The wirings of the N control signals after wiring change are made irregular over the M delay circuits. This configuration changes a matching relationship between the N control signals and the N memory cells in each delay circuit, and makes the use starting stage numbers irregular. The number of delay circuits that are required to change wiring from among the M delay circuits is normally M−1. However, the number of delay circuits which are subject to wiring change is an arbitrary number unless the wirings of the N control signals are regular over the M delay circuits. The wiring change may be performed by hardware. The N control signals are, for example, N write control signals and/or N read control signals.

In an embodiment, each wiring change circuit is a selection circuit to which the N control signals before wiring change, and the N control signals after wiring change, which are shifted from the N control signals before wiring change as much as a predetermined number of stages, are input. The selection circuit selects the N control signals before wiring change in a non-shift mode, and selects the N control signals after wiring change in a shift mode. According to this configuration, it is possible to select the non-shift mode or the shift mode according to the presence or absence and the like of a noise after addition.

The ultrasonic diagnosis device according to an embodiment includes a vibration element array which is formed of a plurality of vibration elements two-dimensionally wired, and has a plurality of sub arrays two-dimensionally wired with respect to the vibration element array. The M delay circuits are connected to the sub arrays on a sub array basis, and the wirings of the N control signals after wiring change are made irregular over the plurality of sub arrays. According to this configuration, noise generation timings can be dispersed over the plurality of sub arrays (over the plurality of sub beamformers).

An electronic circuit according to an embodiment includes M delay circuits, an adding circuit, and a control unit which are installed in an ultrasonic probe. Each delay circuit includes a memory cell array which is formed of N memory cells from a first stage to an N-th stage. The adding circuit adds M reception signals output from the M delay circuits. The control unit controls a cyclic operation of the memory cell array according to a set delay time in each delay circuit. Conditions of the cyclic operations of the M memory cell arrays are made irregular, such that use starting stage numbers in the M memory cell arrays included in the M delay circuits are different. Accordingly, since output timings of noises from the respective memory cell arrays become irregular, an increase of a noise in the adding circuit is avoided.

### (2) Details of Embodiments

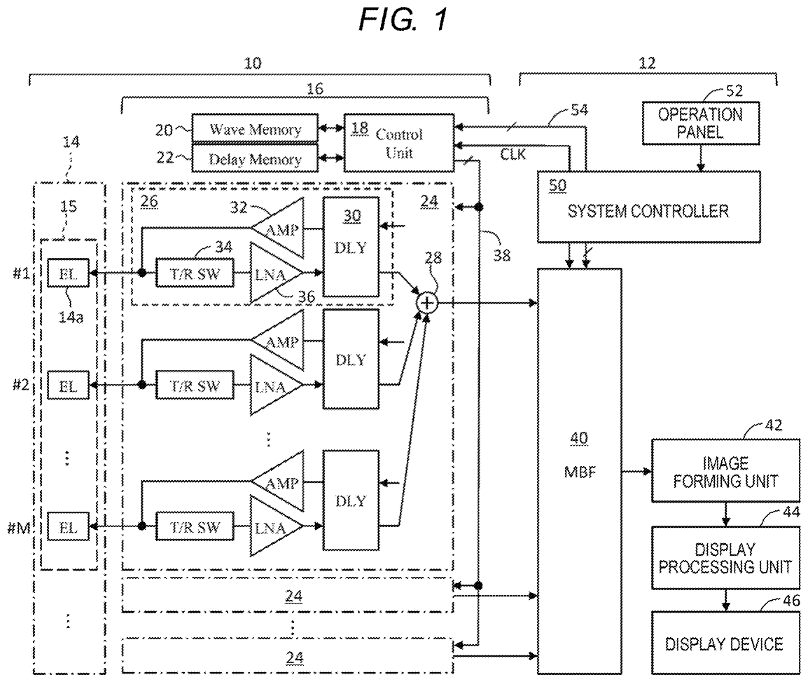

In FIG. 1, a configuration of an ultrasonic diagnosis device according to an embodiment is illustrated as a block diagram. The ultrasonic diagnosis device may be a medical device which is installed in a medical institution such as a hospital or the like, and forms an ultrasonic image by transmitting and receiving ultrasonic waves to and from a living body (examinee).

The ultrasonic diagnosis device generally includes an ultrasonic probe 10 and a device main body 12. The ultrasonic probe 10 is a so-called 3D probe, and includes a two-dimensional vibration element array 14 and an electronic circuit 16. The two-dimensional vibration element array 14 is formed of thousands, tens of thousands, or hundreds of thousands of vibration elements 14a two-dimensionally wired. A plurality of sub arrays 15 are set in the two-dimensional vibration element array 14. Each sub array 15 forms a processing unit in main beamforming. Sub beamforming is applied in each sub array 15. Each sub array 15 is configured with M vibration elements from a first element (#1) to an M-th element (# M) in the illustrated example (Although the vibration elements are linearly arranged in FIG. 1, they may be two-dimensionally wired in reality). Sub beamforming of multiple steps may be performed in the ultrasonic probe 10.

The electronic circuit 16 includes one or a plurality of semiconductor integrated circuits. Specifically, the electronic circuit 16 includes a plurality of sub beamformers 24, a control unit (probe control unit) 18, a waveform memory 20, and a delay data memory 22. Each sub beamformer 24 generates a plurality of transmission signals delay-processed when transmitting, and provides the transmission signals to the plurality of vibration elements in parallel. Each sub beamformer 24 generates a sub beamforming signal by delay-processing a plurality of reception signals from the plurality of vibration elements when receiving, and outputs the sub beamforming signal to the device main body 12.

Specifically, each sub beamformer 24 includes a plurality of transceivers 26, an adding circuit 28, etc. The plurality of transceivers 26 are connected to the plurality of vibration elements constituting the sub array 15 with a one-to-one relationship. Each transceiver 26 includes a delay circuit 30 including a memory cell array. Furthermore, each transceiver 26 includes a transmission amplifier 32, a transmission and reception conversion switch 34, and a reception amplifier (linear amplifier) 36. Instead of the transmission amplifier 32, a pulsar may be installed. The delay circuit 30 generates a delay-processed transmission signal when transmitting, and delay-processes a reception signal when receiving. That is, the delay circuit 30 is a circuit for both transmission and reception.

The control unit 18 is a control circuit as a local controller to be controlled by a system controller 50, which will be described below. The control unit 18 controls an operation of each sub beamformer 24, and for example, controls delay processing at each sub beamformer 24. To achieve this, a control signal 38 is applied to each sub beamformer 24 from the control unit 18.

The waveform memory 20 stores waveform data constituting a transmission signal. The waveform data is transmitted to each sub beamformer 24 if necessary. The delay data memory 22 stores delay data transmitted from the system controller 50. The delay data may be generated in the control unit 18. The control unit 18 transmits the delay data to each sub beamformer 24, or controls each sub beamformer 24 according to the delay data. The configuration of the electronic circuit 16 illustrated in FIG. 1 is merely an example. The ultrasonic probe 10 may be, for example, a body surface contact type probe or a body cavity insertion type probe. In the illustrated configuration example, the ultrasonic probe 10 and the device main body 12 are connected to each other by a cable.

Hereinafter, the device main body 12 will be described. A main beamformer 40 is installed in the device main body 12 as an electronic circuit. A plurality of sub beamforming signals (sub array reception signals) output from the plurality of sub beamformers are input to the main beamformer 40. The main beamformer 40 applies phase regulation and addition (delaying and addition) to these signals, and accordingly, generates beam data. For example, one volume data is configured with a plurality of frame data. One frame data is configured with a plurality of beam data. One beam data is configured with a plurality of echo data arranged in a depth direction.

The image forming unit 42 is configured with a processor which forms a tomographic image as a two-dimensional ultrasonic image based on the frame data, or forms a three-dimensional ultrasonic image based on the volume data. The three-dimensional ultrasonic image is an ultrasonic image that represents tissue stereographically. As a rendering method to achieve this, a volume rendering method, a surface rendering method, etc. are known. The ultrasonic image may be formed based on Doppler information. Data of the ultrasonic image formed in the image forming unit 42 is transmitted to a display device 46 via a display processing unit 44. The ultrasonic image is displayed on the display device 46. The display processing unit 44 is configured with a processor including an image synthesis function, a color operation function, a graphic image generation function, etc. The display device 46 may be configured with a liquid crystal display device, an organic EL display device, or others.

The system controller 50 controls an operation of each element illustrated in FIG. 1, and includes transmission and reception control, particularly, control of the control unit 18 in the ultrasonic probe 10. The system controller 50 is configured with a CPU and an operation program. An operation panel 52 is connected to the system controller 50. The operation panel 52 is an input device which includes a plurality of switches, a plurality of buttons, a trackball, a keyboard, or the like.

Control data 54 is transmitted to the control unit 18 in the ultrasonic probe 10 from the system controller 50. The control unit 18 controls each element in the ultrasonic probe 10, particularly, each sub beamformer 24, according to the control data. A clock is provided to the control unit 18 from the system controller 50.

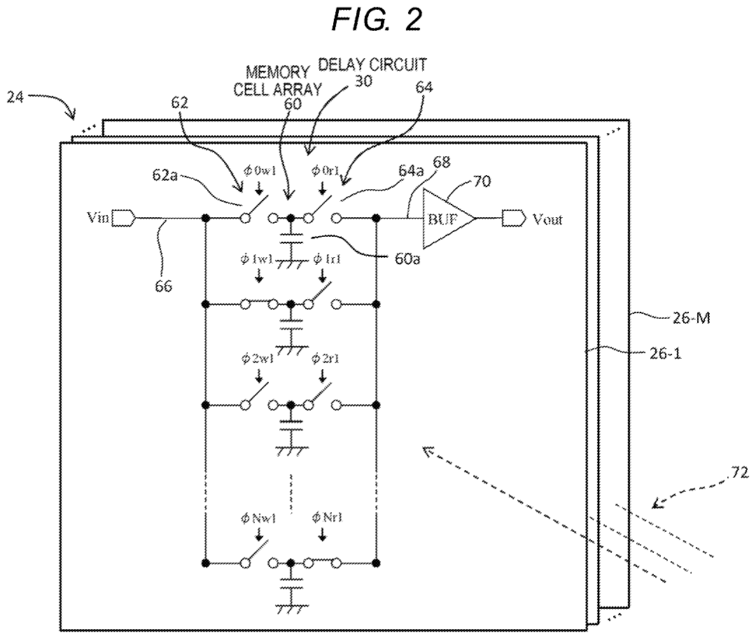

In FIG. 2, the sub beamformer 24 is illustrated. The sub beamformer 24 includes M transceivers 26-1 to 26-M. The transceivers have the same configuration. Herein, the transceiver 26-1 is adopted and a configuration thereof will be described.

The transceiver 26-1 includes the delay circuit 30. The delay circuit 30 includes a memory cell array 60, and the memory cell array 60 is formed of N memory cells 60a installed in parallel. Each memory cell 60a is configured with, for example, an analogue memory (capacitor) 60a. In other words, each memory cell 60a includes one pair of switches 62a, 64a installed at the front and the rear thereof, and functions as a sample and hold (S&H) circuit. A switch array 62 is installed at the front stage of the memory cell array 60, and is formed of N switches 62a. A switch array 64 is installed at the rear stage of the memory cell array 60, and is also formed of N switches 64a.

An input signal 66 is stored, for example, in the k-th memory cell selected by the switch array 62. After a set delay time, a signal is read out from the k-th memory cell by an operation of the switch array 64, and is output to the outside via a buffer 70 as an output signal 68. After a signal is written on the k-th memory cell, a signal is written on the k+1-th memory cell, and, after the set delay time, a signal is read out from the k+1-th memory cell, and is output in the same way as described above. The memory cell array 60 is cyclically used like a ring memory, and the respective signals are delay-processed. In other words, the memory cell array performs a cyclic operation. When a signal is received, a so-called reception dynamic focus is performed by using the delay circuit 30.

Operations of the switch array 62 and the switch array 64 are controlled by the control unit illustrated in FIG. 1. In FIG. 2, control signals for controlling the operations of the respective switches are expressed by sign ϕ. Subscripts 1 to N following ϕ indicate numbers (stage numbers) of the memory cells, and letters w and r following the subscripts indicate write and read, respectively. Numbers following these indicate numbers of the sub beamformers. The numbers are 1 to M. M reception signals output from the M transceivers 26-1 to 26-M are added by the adding circuit, and as a result, a sub beamforming signal is generated.

When common noises 72 transversally enter memory cells of a specific stage number in the M memory cell arrays, or common noises occur in the memory cells of the specific stage number, a great periodic noise occurs as a result of adding M noises in the adding circuit. This degrades an S/N ratio, and furthermore, degrades image quality of an ultrasonic image. Such a phenomenon is easy to occur when the plurality of sub beamformers are established on a semiconductor integrated circuit. The noise occurs due to variation of a circuit characteristic, parasitic capacitance attributable to a circuit layout, crosstalk, or the like.

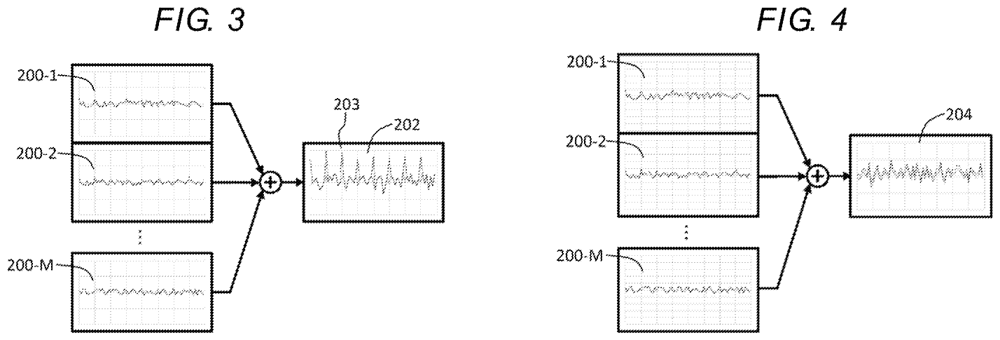

FIG. 3 illustrates such a phenomenon. When reception signals 200-1 to 200-M from a first signal to an M-th signal are added in the adding circuit, a comparatively great periodic noise 203 occurs in a signal 202 after addition. In the description of the present application, the same reference numerals are used for the elements described above in each drawing, and a description thereof is omitted.

According to a control method according to an embodiment, the use starting stage numbers of the M memory cell arrays are controlled such that time phases of the M noises are dispersed at the adding step, as will be described below in detail. As a result, a noise in a signal 204 after addition becomes inconspicuous.

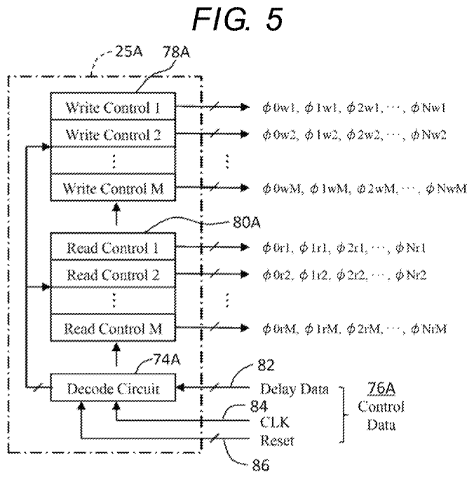

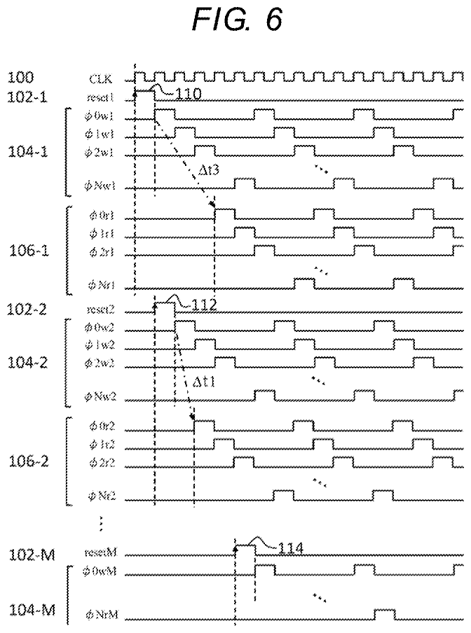

FIGS. 5 and 6 illustrate the first embodiment. In the first embodiment, the use starting stage numbers are made irregular over the M delay circuits (M memory cell arrays) by making timings of M reset signals (start triggers) to be applied to the M delay circuits irregular.

In FIG. 5, a control circuit 25A is illustrated. In the control unit illustrated in FIG. 1, the control circuit 25A illustrated in FIG. 5 is installed for each sub beamformer. The control circuit 25A may be installed in the sub beamformer. The control circuit 25A includes a decode circuit 74A, a write control block 78A, and a read control block 80A. Control data 76A is applied to the decode circuit 74A from a core module in the device main body or the control unit. In the illustrated example, the control data 76A includes delay data 82, a clock 84, and reset data 86.

The write control block 78A is formed of M write control modules corresponding to the M delay circuits. Each of the write control modules generates N write control signals to be applied to the memory cell array formed of the N memory cells. The read control block 80A is formed of M read control modules corresponding to the M delay circuits. Each of the read control modules generates N read control signals to be applied to the memory cell array formed of the N memory cells. The write control block 78A and the read control block 80A operate according to the control data provided from the decode circuit 74A.

In the first embodiment, the reset data 86 is configured with M reset signals, and the M reset signals are applied to the M write control modules and the M read control modules. Each of the reset signals functions as a start trigger. M reset timings according to the M reset signals are made irregular, and it is possible to make the use starting stage number different in each delay circuit, by applying such M reset signals to the M write control modules and the M read control modules in parallel.

FIG. 6 illustrates an operation of the sub beamformer in the first embodiment. The M delay circuits (that is, M memory cell arrays) are included in the sub beamformer. Reference numeral 100 indicates a clock. Reference numeral 102-1 indicates a first reset signal, that is, a first start trigger, for regulating the operation of the first delay circuit. 102-2 indicates a first reset signal, that is, a first start trigger, for regulating the operation of the second delay circuit. Reference numeral 102-M indicates an M-th reset signal, that is, an M-th start trigger, for regulating the operation of the last M-th delay circuit. Time phases of the reset signals 102-1, 102-2, 102-M are different from one another, and are dispersed along a time axis. In the example illustrated in FIG. 6, the timings of the reset signals from the first signal to the M-th signal are shifted in sequence, but may be shifted irregularly or randomly (pseudo-randomly).

Reference numeral 104-1 indicates N write control signals applied to the first delay circuit. Reference numeral 106-1 indicates N read control signals applied to the first delay circuit. Any of these signals is a signal for controlling on and off of the switches installed at the front and the rear of each memory cell. The N write control signals 104-1 and the N read control signals 106-1 are generated while using the first reset signal 102-1 (specifically, a reset pulse 110) as a temporal reference. A delay time regarding the memory cell of the first stage in the first memory cell array is indicated by Δt3.

Reference numeral 104-2 indicates N write control signals applied to the second delay circuit. Reference numeral 106-2 indicates N read control signals applied to the second delay circuit. The N write control signals 104-2 and the N read control signals 106-2 are generated while using the second reset signal 102-2 (specifically, a reset pulse 112) as a temporal reference. A delay time regarding the memory cell of the first stage in the second memory cell array is indicated by Δt1.

Reference numeral 104-M indicates N write control signals applied to the M-th delay circuit (However, a portion thereof is illustrated in the drawing). The N write control signals 104-M applied to the M-th delay circuit, and N read control signals applied to the M-th delay circuit are generated by using the M-th reset signal 102-M (specifically, a reset pulse 114) as a temporal reference.

According to the first embodiment described above, the use starting stage numbers in the M memory cell arrays can be dispersed in each of the sub beamformers. Accordingly, when delay times between the plurality of delay circuits become regular, a problem that a plurality of signals are simultaneously read out from the memory cells of the same stage number in each time phase, and accordingly, a noise increases at the adding step can be effectively suppressed.

The M reset signals are generated when transmission and reception start, are generated every time a reception beam is formed, or are generated every time the delay time is converted. The M reset signals may be generated at the other timing.

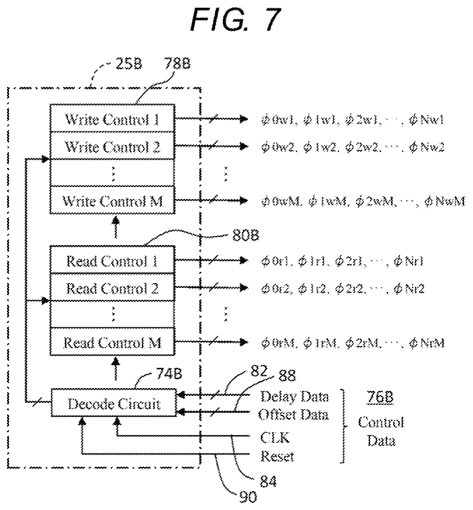

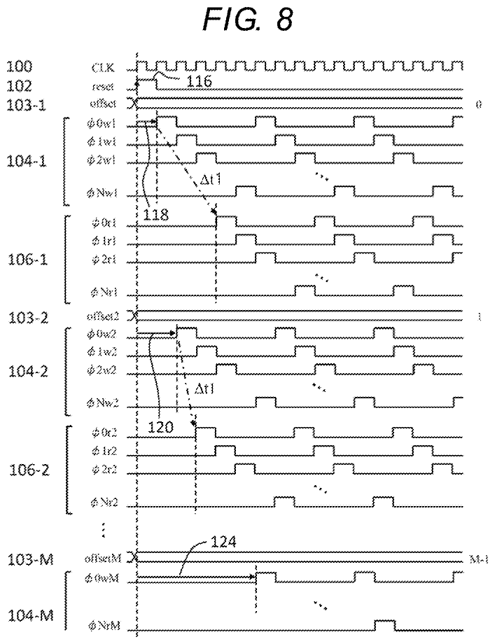

FIGS. 7 and 8 illustrate the second embodiment. In the second embodiment, the use starting stage numbers are made irregular over the M delay circuits (M memory cell arrays) by making M offsets (shift mount of a starting memory cell) for regulating the operations of the M delay circuits irregular.

FIG. 7 illustrates a control circuit 25B. The control circuit 25B illustrated in FIG. 7 is installed in each sub beamformer. As described above, the control circuit 25B may be installed in the sub beamformer. The control circuit 25B includes a decode circuit 74B, a write control block 78B, and a read control block 80B. Control data 76B is applied to the decode circuit 74B from a core module in the device main body or the control unit. The control data 76B includes delay data 82, a clock 84, and a reset signal 90, and further includes offset data 88 in the illustrated example.

The write control block 78B basically includes the same configuration as the write control block 78A illustrated in FIG. 5. The read control block 80B basically includes the same configuration as the read control block 80A illustrated in FIG. 5.

In the second embodiment, the reset signal 90 is one common signal with respect to the M delay circuits. On the other hand, the offset data 88 is configured with M offsets (offset signals), and the M offsets are applied to M write control modules and M read control modules in parallel.

FIG. 8 illustrates an operation of the sub beamformer in the second embodiment. As described above, a reset signal 102 is a common signal with respect to the M delay circuits (M memory cell arrays). In reality, the M delay circuits are synchronized in parallel and operates by using a reset pulse 106 of the reset signal 102 as a temporal reference.

Reference numeral 103-1 indicates a first offset (offset value: 0), reference numeral 103-2 indicates a second offset (offset value: 1), and reference numeral 103-M indicates an M-th offset (offset value: M−1). Each of the offsets is for regulating a delay amount of write starting timing from a reference time defined by a reset pulse. The first blank period increases as it approaches the M-th delay circuit. However, since such a blank period is not really used, the blank period does not matter.

According to the second embodiment described above, it is possible to make the use starting stage numbers between the M memory cell arrays irregular, by using the ununiform offsets in each sub beamformer. Accordingly, a problem that a noise increases at the adding step can be solved or reduced. In the example illustrated in FIG. 8, the offsets from the first offset to the M-th offset increase linearly, but the offsets may be shifted irregularly or randomly.

The M offsets are generated, for example, when transmission and reception start, are generated every time a reception beam is formed, or are generated every time the delay time is converted. The M reset signals may be generated at the other timing.

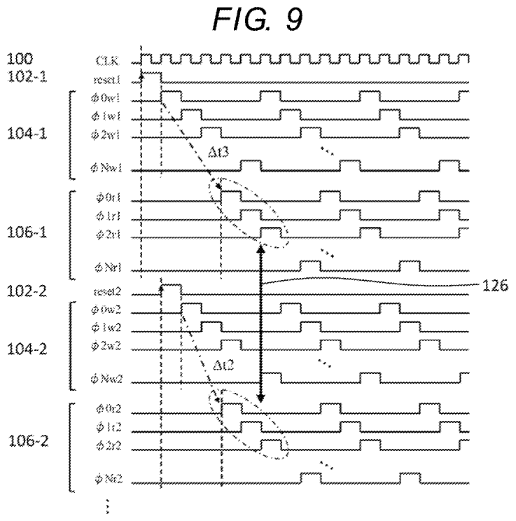

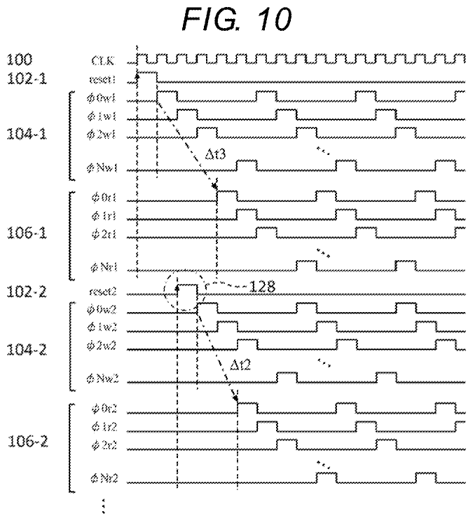

A variation of the first embodiment described above will be described by using FIGS. 9 and 10. As shown in FIG. 9, according to the first embodiment, it is possible to make the use starting stage numbers of the M memory cell arrays irregular by shifting timings of the start triggers in sequence. However, according to the delay time given to each memory cell, a phenomenon that signals are simultaneously read out from the plurality of memory cells belonging to the same stage number may occur. For example, in FIG. 9, a delay amount set for the memory cell of the first stage in the first memory cell array is Δt3. A delay amount set for the memory cell of the first stage in the second memory cell array is Δt2. As indicated by reference numeral 126, timings of the start triggers between the two memory cell arrays are different, but signals are simultaneously read out from the two first-stage memory cells in relation to the delay amount.

In this case, simultaneous reading from the same stage number can be avoided by correcting the timing of the start trigger as shown in FIG. 10. That is, when simultaneous reading of more than a predetermined number from the same stage number is predicted based on the delay amount, the simultaneous reading can be avoided by correcting the timing of the start trigger. Since the same problem may arise in the second embodiment, the offset may be corrected according to the delay time to avoid such a problem.

However, according to such a variation, an operation and a control may become complicated. Therefore, when there is a margin in processing of the electronic circuit in the ultrasonic probe, it is desirable to adopt the variation.

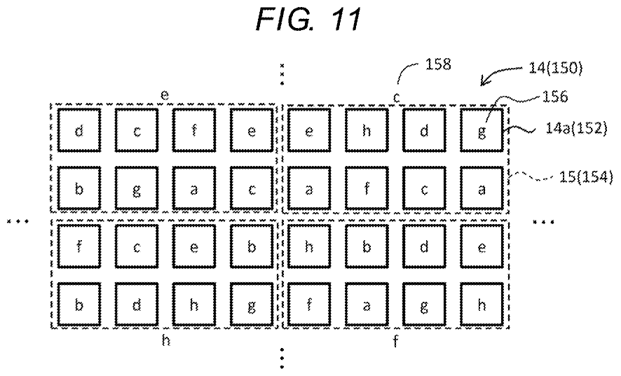

Hereinafter, the third embodiment will be described based on FIGS. 11 to 14. The first and second embodiments aim at dispersing in the process of generating N control signals, and the third embodiment aims at dispersing after generating N control signals, that is, post dispersion, and in particular, aims post dispersion using a hardware circuit.

FIG. 11 illustrates a part of a vibration element array 14. The vibration element array 14 is configured with a plurality of vibration elements 14a, and a plurality of transceivers 152 are connected to the plurality of vibration elements 14a. A transceiver array 150 is configured with the plurality of transceivers 152. A plurality of sub arrays 15 are set for the vibration element array 14, and a sub beamformer 154 is installed for each sub array 15. Each sub beamformer 154 includes the plurality of transceivers 152 and an adding circuit.

In the third embodiment, wiring shift numbers (parameters for making irregularity), which will be described below, are randomly set over the plurality of sub arrays 15 (that is, the plurality of sub beamformers 154). In addition, wiring shift numbers are randomly set in each sub array 15 on the basis of the vibration element 14a. Symbols a to h shown in reference numerals 156 and 158 indicate wiring shift numbers which are different. 0 may be included as a wire shift number. In reality, when the memory cell array is configured with N memory cells, N−1 wiring shift numbers (numerical values of 1 to N−1) may be selected. Reference numeral 156 indicates a wiring shift number on the basis of an element. In the illustrated example, the wiring shift numbers are randomly set between the sub arrays and in the sub array. Reference numeral 158 indicates a wiring shift number in delaying and addition at the second stage when two-stage sub beamforming is performed. To prevent an increase of a noise even in the delaying and addition, the wiring shift numbers are set spatially randomly.

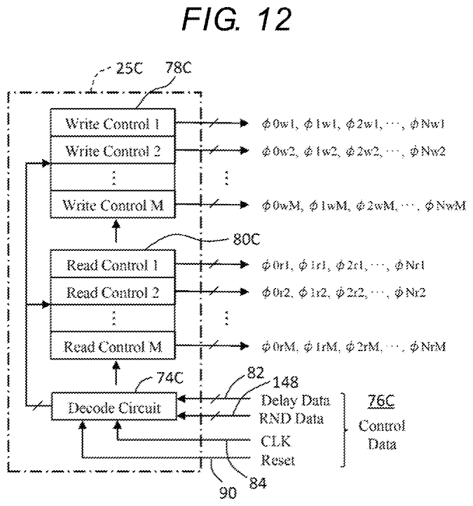

In FIG. 12, a control circuit 25C of the third embodiment is illustrated. The control circuit 25C illustrated in FIG. 12 is installed for each sub beamformer. As described above, the control circuit 25C may be installed in the sub beamformer. The control circuit 25C includes a decode circuit 74C, a write control block 78C, and a read control block 80C. Control data 76C is applied to the decode circuit 74C. The control data 76C includes delay data 82, a clock 84, and a reset signal 90 in the illustrated example, and a mode selection signal 148 is included therein. The write control block 78C basically has the same configuration as the write control block 78A shown in FIG. 5. The read control block 80C basically has the same configuration as the read control block 80A shown in FIG. 5.

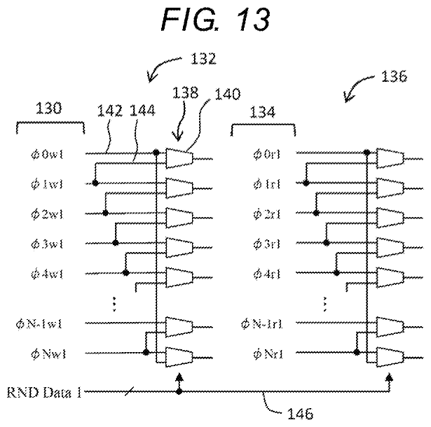

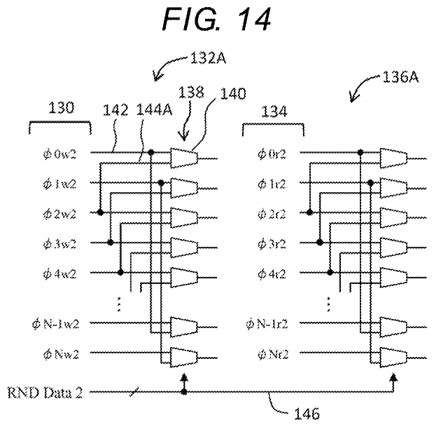

In the third embodiment, a wiring change unit illustrated in FIGS. 13 and 14 is installed at the rear stage of the control circuit 25C or as an output unit thereof. In FIG. 13, the wiring change unit having the wiring shift number of 1 is illustrated, and the wiring change unit is configured with a write control wiring change circuit 132 and a read control wiring change circuit 134. Such a wiring change unit is installed, for example, in the first sub beamformer.

The write control wiring change circuit 132 is a circuit which selectively outputs N write control signals (see reference numeral 130) or N write control signals in which one wiring is shifted up. Specifically, the write control wiring change circuit 132 includes a selection circuit 138, which is configured with N selectors 140. The N write control signals are input to the N selectors 140 as they are, or the N write control signals after wiring change, which are configured by shifting up wirings of the N write control signals by one stage. The N selectors 140 select any one of the two types of the N write control signals input, according to a mode selection signal 146. The read control wiring change circuit 134 is a circuit which selectively outputs N read control signals (see reference numeral 140) or N read control signals in which one wiring is shifted up. The read control wiring change circuit has the same configuration as the write control wiring change circuit 132 described above.

In FIG. 14, a wiring change unit having the wiring shift number of 2 is illustrated. The wiring change unit is configured with a write control wiring change circuit 132 and a read control wiring change circuit 134. Such a wiring change unit is installed, for example, in the second sub beamformer. Other wiring change units may be configured in the same way as described above. For example, the wiring change unit illustrated in FIG. 13 corresponds to symbol “a” illustrated in FIG. 11. The wiring change unit illustrated in FIG. 14 corresponds to symbol “b” illustrated in FIG. 11. In addition, when the wiring shift number is 0, there is no need to install the wiring change unit. Typically, M−1 wiring change units are installed with respect to the M memory cell arrays. When the wiring shift number is randomly set, M wiring change units may be installed with respect to the M memory cell arrays, and the number of omitted wiring change units may be two or more.

In the third embodiment, it is possible to avoid or reduce the problem that a noise increases at the adding step on the basis of the sub beamformer. Furthermore, in the third embodiment, the problem of an increase of a noise between the sub arrays (increase of a noise at the second stage addition) can also be avoided or reduced. However, the technology described as the first or second embodiment may be applied to beamforming of multiple stages. In addition, in the third embodiment, wiring change is performed by hardware, but may be performed by software.

According to the first, second, and third embodiments described above, when the plurality of memory cell arrays are operated in parallel, the problem that noises are simultaneously output from the plurality of memory cells of a specific stage, and are added can be solved or reduced. For example, by dispersing noise occurring timings at N stages, the intensity of a noise can be adjusted to (N)^{1/2} or a value close thereto. In this case, there is no need to additionally arrange a memory cell, and from this aspect, complexity of control and configuration can be avoided.

What is claimed is:

1. An ultrasonic diagnosis device comprising:
M delay circuits configured to delay M reception signals, each delay circuit comprising a memory cell array which is formed of N memory cells from a first stage to an N-th stage, the M delay circuits operating in parallel;
an adding circuit configured to add the M reception signals output from the M delay circuits; and
a control unit configured to control a cyclic operation of the memory cell array according to a set delay time in each delay circuit, wherein
conditions of the cyclic operations of the M memory cell arrays are made irregular, such that use starting stage numbers in the M memory cell arrays included in the M delay circuits are different.

2. The ultrasonic diagnosis device of claim 1, wherein
in each of the delay circuits, the cyclic operation of the memory cell array starts according to a start trigger, and
the control unit is configured to make timings of M start triggers to be applied to the M delay circuits irregular.

3. The ultrasonic diagnosis device of claim 2, wherein
the control unit is configured to correct the timings of the M start triggers according to the delay time set in each of the delay circuits.

4. The ultrasonic diagnosis device of claim 1, wherein
in each of the delay circuits, the cyclic operation of the memory cell array starts from a memory cell of a stage number corresponding to an offset, and
the control unit is configured to make M offsets to be applied to the M delay circuits irregular.

5. The ultrasonic diagnosis device of claim 4, wherein
the control unit is configured to correct the M offsets according to the delay time set in each of the delay circuits.

6. The ultrasonic diagnosis device of claim 1, wherein
the control unit includes:
a generation circuit installed in each of the delay circuits to generate N control signals to be applied to the N memory cells; and
a wiring change circuit configured to change wirings of the N control signals in each delay circuit which is required to change wiring from among the M delay circuits, and to output N control signals after wiring change, and
the wirings of the N control signals after the wiring change are made irregular over the M delay circuits.

7. The ultrasonic diagnosis device of claim 6, wherein
each of the wiring change circuits is a selection circuit to which N control signals before wiring change, and N control signals after wiring change which are shifted from the N control signals before the wiring change as much as a predetermined number of stages, are input, and
the selection circuit is configured to select the N control signals before the wiring change in a non-shift mode, and to select the N control signals after the wiring change in a shift mode.

8. The ultrasonic diagnosis device of claim 6, further comprising:
a vibration element array which is formed of a plurality of vibration elements two-dimensionally wired, wherein
a plurality of sub arrays two-dimensionally wired are set with respect to the vibration element array,
the M delay circuits are connected to the sub array based on the sub array, and
the wirings of the N control signals after the wiring change are made irregular over the plurality of sub arrays.

9. An electronic circuit comprising:
M delay circuits installed in an ultrasonic probe to delay M reception signals, each delay circuit comprising a memory cell array which is formed of N memory cells from a first stage to an N-th stage, the M delay circuits operating in parallel;
an adding circuit installed in the ultrasonic probe to add the M reception signals output from the M delay circuits; and
a control unit installed in the ultrasonic probe to control a cyclic operation of the memory cell array according to a set delay time in each of the delay circuits, wherein
conditions of the cyclic operations of the M memory cell arrays are made irregular, such that use starting stage numbers in the M memory cell arrays included in the M delay circuits are different.
